(19) United States
(12) Patent Application Publication
(10) Pub. No.: US 2008/0140203 A1
(43) Pub. Date: Jun. 12, 2008

(54) Intervertebral disc support coil and screw applicator

(76) Inventor: Reginald Davis, Cockeysville, MD (US)

Correspondence Address: Ober, Kaler, Grimes & Shriver, 120 East Baltimore Street, Baltimore, MD, 21202-1643, US

(21) Appl. No.: 11/977,438

(22) Filed: Oct. 24, 2007

Related U.S. Application Data
(60) Provisional application No. 60/854,292, filed on Oct. 24, 2006.

(51) Int. Cl. A61F 2/44

(52) U.S. Cl. 623/17.13

(57) ABSTRACT

A spinal intervertebral support coil sized to fit inside the intervertebral sac between adjacent vertebral bones of the spine and formed as a helical spring of high-resiliency material. The support coil is adapted for corkscrew insertion within an intervertebral disk sack between opposing disk bodies for additional (augmenting) support thereof by interposing a predetermined bias between opposing disk bodies, yet allowing a limited degree of rotational movement, torsional movement, and axial movement in a compressive direction for full displacement, rotation, subluxation, flexion, and extension of the vertebral bodies. An insertion tool is also disclosed for corkscrew insertion of the support coil, and this includes a handle and body with a rotating rod keyed to the support coil. A method for insertion of the spinal intervertebral support coil is also described.

### CROSS-REFERENCE TO RELATED APPLICATION(S)

The present application derives priority from U.S. provisional application Ser. No. 60/854,292 filed 24 Oct. 2006.

### BACKGROUND OF THE INVENTION

1. Field of the Invention

The present invention relates to intervertebral disc supports and, more particularly, to an intervertebral disc support coil and screw-type applicator for minimally-invasive insertion of the coil between vertebrae.

2. Description of the Background

Intervertebral discs are circular structures that function as cushions between spinal vertebrae. Each intervertebral disc contains a nucleus (center) surrounded by a sack of fibrocartilage (fibrous, connective tissue), rich in collagens (fibrous protein). The intervertebral disc is a vital component of the functional spinal unit. They maintain space between adjacent vertebral bodies, and absorb impact. Deterioration of an intervertebral disc may result from disease, trauma or aging, and symptoms include limited mobility, and severe pain. Trauma damage may include ruptures, tears, prolapse, herniations, and other injuries that cause pain and reduce strength and function. For example, a herniated disc occurs when the outer sack partially ruptures and the interior of the sack expands, pushing part of the disc into the spinal canal. This condition is also known as a slipped disc, an intervertebral disc hernia, a herniated intervertebral disc, and a herniated nucleus pulposus.

Intervertebral disc degeneration and vertebral trauma are very common, and other disease conditions may lead to defects such as tumors, necrosis, and endocrine conditions. Estimated health care costs of treating back pain in the United States exceed $60 billion annually, and pose substantial costs in the form of disability payments, workers' compensation and lost wages.

There are a few non-operative therapies including rest, analgesics, physical therapy, heat, and manipulation. These treatments are usually only helpful for milder conditions. For more severe cases surgical options include discectomy, fusion, and a combination of the two. These surgical options are highly invasive and require prolonged hospitalization and recovery.

There is most clearly a need to treat vertebral and intervertebral injury and disease using minimally invasive techniques. Various implants, surgical meshes, patches, barriers, tissue scaffolds and the like may be used to treat intervertebral discs and are known in the art. Surgical repair meshes are used throughout the body to treat and repair damaged tissue structures such as herniated discs.

There are numerous artificial intervertebral discs for replacing a part or all of a removed disc. Many of these use soft cushions that function similar to the disc they replace. Examples of such disc replacements are disclosed in U.S. Pat. Nos. 5,702,450 and 5,035,716. A ball and socket arrangement has also been attempted, but these lack the natural motion of the intervertebral disc. Moreover, dislocation and wear are concerns with these disc replacements.

There are also a variety of spring discs that employ springs sandwiched between metal endplates. For example, U.S. Pat. No. 5,458,642 to Beer et al. issued Oct. 17, 1995 shows a synthetic intervertebral disc for implantation in the human body. The disc is comprised of disc-shaped plates 11 joined by springs along the inside. The plates have oval-like cutouts in their centers for a compressible polymeric core 12 to protrude from on top and bottom. An elastomeric covering 14 encircles the area between the plates and is connected to the plates 11 on top and bottom to prevent body tissues from interfering with the movement of the springs 13. The spring system distributes forces acting on the disc between the springs and allows normal movement of the vertebrae during flexion and extension of the spine in any direction. While the foregoing device may approximate normal motion, it is a complex assembly and is not suitable for minimally-invasive in-body implantation. The vertebral bodies must be removed, the disc-shaped plates 11 attached, and the entire assembly implanted.

It would be much more advantageous to provide a spring-like intervertebral disc support coil designed not to replace a disc, but only to reinforce it and restore normal functioning. A spring is a helix, and a helix can be inserted in a cork-screw manner. Thus, springs lend themselves to minimally-invasive implantation. For example, for other medical specialties there are numerous helical screw-insertion type staples and tacks for fastening tissue. U.S. patent application no. 20060036265 by Dant discloses a helical suturing device for repairing a tear in an annulus fibrosus of a spinal disc. However, this is only for suturing, not supporting, and the suture eventually dissolves.

It would be greatly advantageous to provide a helical corkscrew-type implant for intervertebral disc support, and minimally invasive applicator therefor.

### SUMMARY OF THE INVENTION

It is therefore an object of the present invention to provide a helical support coil for effectively augmenting the existing sack of fibrocartilage (rather than replacing it) without sacrificing any range of motion.

It is another object to provide a helical corkscrew-type implant for and minimally invasive applicator to accomplish the foregoing.

It is another object to provide an applicator as described above with one or more lumens extending there through for irrigation and/or visualization of the procedure.

In accordance with the foregoing objects, the present invention is a flexible and self-expanding tissue support structure formed as a helix that can be screw-inserted into the existing intervertebral disk tissue structures in a screw-insertion manner to serve as a resilient supporting or reinforcing structure. A screw-type insertion tool is also disclosed.

### BRIEF DESCRIPTION OF THE DRAWINGS

Other objects, features, and advantages of the present invention will become more apparent from the following detailed description of the preferred embodiment and certain modifications thereof when taken together with the accompanying drawings in which:

FIG. 1 is a perspective view of the spinal intervertebral support coil 2 according a preferred embodiment of the present invention.

FIG. 2 illustrates the most basic insertion method.

FIG. 3 is an exemplary insertion tool 100 is shown.

FIG. 4 is an exemplary dock 200 for use with the insertion tool 100.

FIG. 5 is an alternative embodiment of an insertion tool 300 which is similar to that of FIG. 3 except to illustrate that the barrel may be curved and/or flexible for easier insertion.

### DETAILED DESCRIPTION OF THE PREFERRED EMBODIMENTS

The present invention is a resilient intervertebral disc support coil formed as a helix to allow screw-insertion into the existing intervertebral disk tissue structure. A screw-type applicator for the disk is also disclosed for minimally-invasive insertion of the coil between vertebrae.

The disc support coil is a much less intrusive alternative to the other two extremes: an artificial intervertebral disc (which replaces a part or all of a removed disc), or full spinal fusion. Rather than replacing or fusing the damaged disk the present device bolsters it, making best use of the existing tissue structure and essentially giving it a new foundation. The present invention also includes the method of insertion and an inserter device, all of which are intended for moderately (but likely not severely) damaged disks.

The intervertebral disc support coil is inserted in a screw-insertion manner to serve as a resilient supporting or reinforcing structure. Once inserted, the intervertebral disc support coil preferably has an expansion capability by water absorption or otherwise, so that the helical coils expand to augment the reinforcing structure.

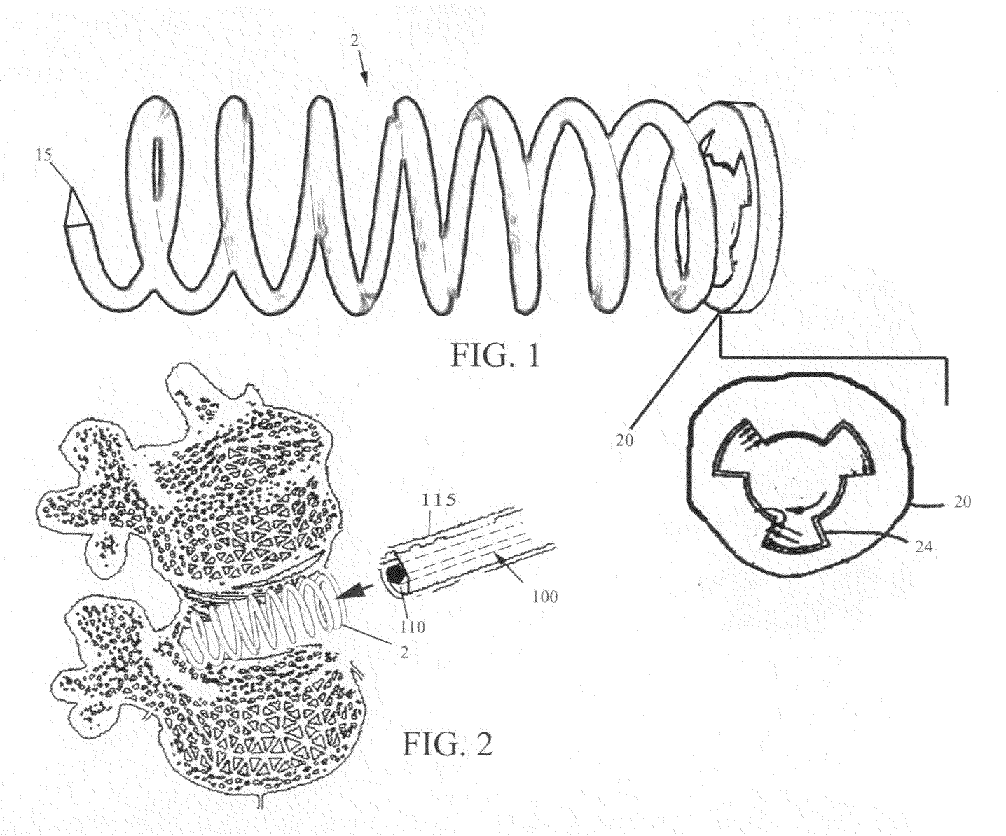

FIG. 1 is a perspective view of the spinal intervertebral support coil 2 according a preferred embodiment of the present invention. The support coil 2 is sized and shaped to fit inside the intervertebral sac between adjacent vertebral bones of the spine. The support coil 2 generally comprises a helical spring having a sharp distal tip 15 formed for tissue penetration.

The support coil 2 may be formed of high-resiliency bio-compatible metal, composite or polymer, and is most preferably formed wholly or partially of a material that gives it an expansion capability once inserted. Hydrogel™ in a desaturated form is one commercially-available material that will suffice. Hydrogel™ comprises a network of polymer chains that are water-insoluble, such that water acts as a dispersion medium. Hydrogels are superabsorbent natural or synthetic polymers and can become saturated to contain over 99% water. In the unsaturated (insertion) form the Hydrogel is rigid enough to withstand screw-insertion, and yet once inserted it attains a degree of flexibility very similar to natural tissue, due to the absorbed water content. Expansion occurs upon absorption and thus gives support coil 2 it's self-expanding capability after insertion. Of course, the support coil 2 must be formed with a predetermined size and shape to fit reasonably closely inside the intervertebral sac occupying a majority of the space between adjacent vertebral bones of the spine, and the unsaturated size must account for the expansion amount after saturation. Thus, the combined factors are calculated to yield a final disk-to-disk bias resembling that of a healthy disk when compressed between the inner surfaces of the respective vertebral bones. On the other hand, the support coil 2 allows a degree of both rotational movement, torsional movement, and axial movement in the compressive direction (due to compression of the coils of the spring). This allows a limited amount of displacement, rotation, subluxation, flexion, and extension of the vertebral bones.

It is also envisioned that a shape memory alloy such as Nitinol™ may be used to achieve the expansion capability. Shape memory alloys “remember” their geometry, such that upon heating it will regain a predetermined shape. The degree of deformation can be tuned by varying the elemental ratios. Thus, a Nitinol™ support coil 2 can be inserted at 70 degree F. in the helical shape illustrated in FIG. 1 and upon reaching body temperature will regain a slightly expanded size and shape to properly bias the adjacent vertebral bones of the spine.

In practice, a surgeon will select from a plurality of different sized intervertebral support coils 2 depending on the particular physiology of the patient. In general, relatively larger intervertebral support coils 2 will be useful in the lumbar region of the spine, smaller sized intervertebral support coils 2 will be useful in the thoracic region of the spine, and still smaller sized intervertebral support coils 2 will be useful in the cervical spine.

By way of example, it is preferred that a height H of an exemplary intervertebral support coil 2 (e.g., measured from the upper to lower extent of coils) is approximately 10 mm, though size will vary somewhat depending on the region of the spine. More particularly, a number of different sized intervertebral support coils 2 are preferably available to the surgeon, such as having a height of between about 8.0 mm to 10.0 mm.

The support coil 2 is equipped with a driver 20 secured or integrally formed at the other end of the device. The driver 20 may be a keyed hub as shown in the lower inset, or any other keyed means capable of cooperation with an inserter (to be described) for screw-insertion. The illustrated embodiment of driver 20 comprises an end-plate having a circular notch defined by a series of axial keyslots 24 evenly spaced around the notch to provide a means for engagement with the screw-insertion tool. One skilled in the art should understand that any suitable keyed hub configuration will suffice for this purpose.

Thus, as shown in FIG. 2, which is a diagram illustrating the most basic insertion method, the support coil 2 is loaded into an insertion tool 100 having a keyed rod 110 rotatably carried within a tubular barrel 115 of the insertion tool 100. The distal end of the keyed rod 110 conforms to the keyslots 24 such that rotation of the rod 110 effects rotation of the support coil 2. The barrel 115 encloses the support coil 2 during insertion into the body.

To insert the support coil 2 between the vertebral bones, an actuator (here obscured) integral to the insertion device 100 rotates the rod 110 and causes rotation of the support coil 2, thereby advancing the coil 2 into the disk. Note that only a small minimally-invasive pinprick-type penetration is needed through the outer disk membrane to facilitate full lengthwise insertion of the support coil 2 into the disk. This is much less intrusive than prior art disk replacement or stabilization methods. If necessary, the actuator in insertion device 100 may also simultaneously or independently push the support coil 2 off the keyed rod 110 to assist with insertion.

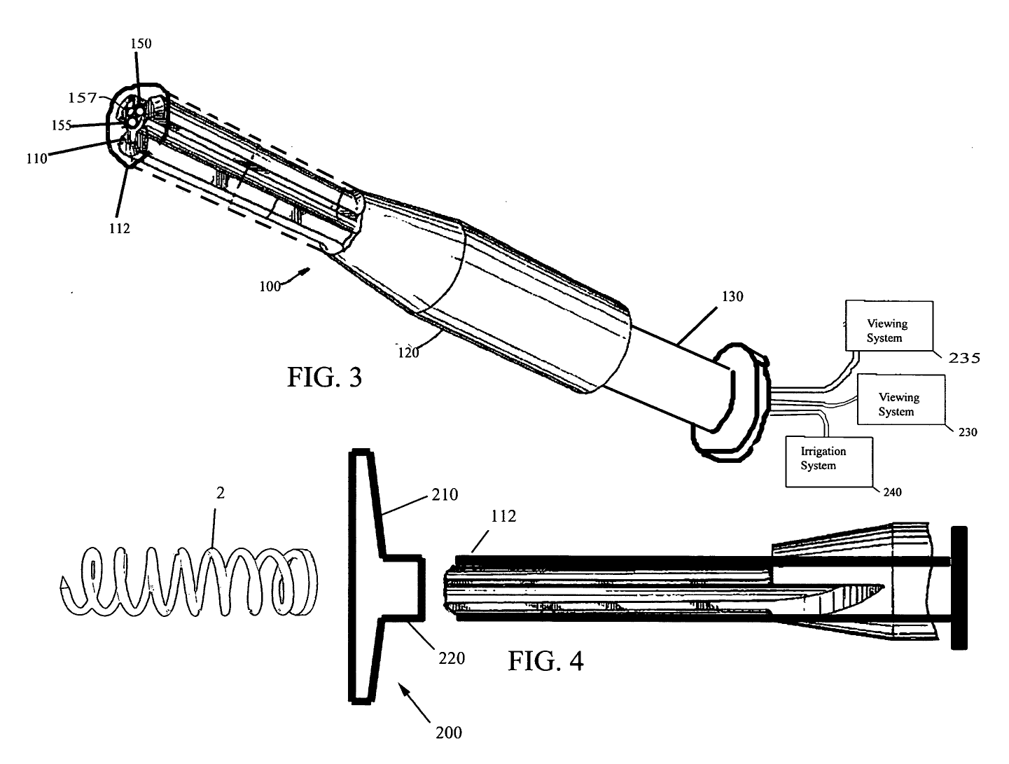

With reference to FIG. 3, the exemplary insertion tool 100 is shown. The insertion tool 100 includes the actuator 130 coupled to the keyed rod 110, a non-rotating body 120, and a non-rotating barrel 112 protruding from the body 120 and covering the rod 110 in a coaxial manner whilst leaving space for insertion of the support coil 2 onto the rod 110. The support coil 2 is inserted onto the rod 110 inside the barrel 112 and simple turning of the actuator 130 turns the rod 110 and likewise the support coil 2 in a cork-screw manner. With tip 15 of support coil 2 embedded in the sack of fibrocartilage (fibrous, connective tissue), the corkscrew motion effectively inserts the support coil fully into the sack between the opposing vertebral bodies. The surgeon can easily manipulate the position of the insertion tool 100 and hence support coil 2 by way of the handle/body 120 in order to manipulate the coil 2 into the intervertebral space, as shown in the inset of FIG. 2.

Note that the insertion tool 100 may include one or more lumens 150, 155 extending through the actuator 130 and the rod 110 and distally outward there from for irrigation, tissue removal and/or visualization of the procedure. In this embodiment the handle is formed with one or more lumens extending there through (here three lumens 150, 155, 157 are shown, and both generally comprising a capillary channel or tube running through the actuator 130 and out from the rod 110 distally. The lumen(s) 150, 155, 157 allow coupling of an irrigation system 240 and/or viewing system 230 (such as an endoscope) to the device, or a laparoscopic tissue resection system for removal of tissue and/or for biopsies to be examined under a microscope. In the case of the irrigation system 240, water or other irrigating fluid may be pumped through the lumen 150 and out from the rod 110 for irrigation or medication of the procedure site. Similarly, in the case of the viewing system 230, an optical fiber may be routed through the lumen 155 and oriented out from the rod 110 (through an embedded lens or the like) for endoscopic viewing of the procedure site. Likewise, for a tissue resection or biopsy system 235, a capillary channel may be routed through the lumen 157 to introduce vacuum suction into the disk, thereby pulling the disk tissue 58 into the lumen 157. Alternatively, an electrosurgical (ablation) type tissue resection system 235 may be employed.

As noted above, it may be desirable to simultaneously or independently push the support coil 2 off the keyed rod 110 to assist with insertion, and this can be readily accomplished by incorporating an annular pusher or piston inside the barrel to forcibly eject the support coil 2.

FIG. 4 illustrates an insertion dock 200 designed for adding stability during insertion of the support coil 2. The insertion dock 200 is an annular member having a flared support 210 for abutting a patients back, and a collar 220 into which the barrel 130 of insertion device 100 docks. The collar 220 has an aperture passing through the entire dock 200. Thus, after an initial entry incision has been made the support coil 2 can be loaded into the insertion device, the dock 200 brought to bear over the incision, and the insertion device 100 coupled into the dock 200 before the insertion procedure as described above. The dock 200 may be adhered to the skin or held tight to provide much greater stability and precision with the instrument 100.

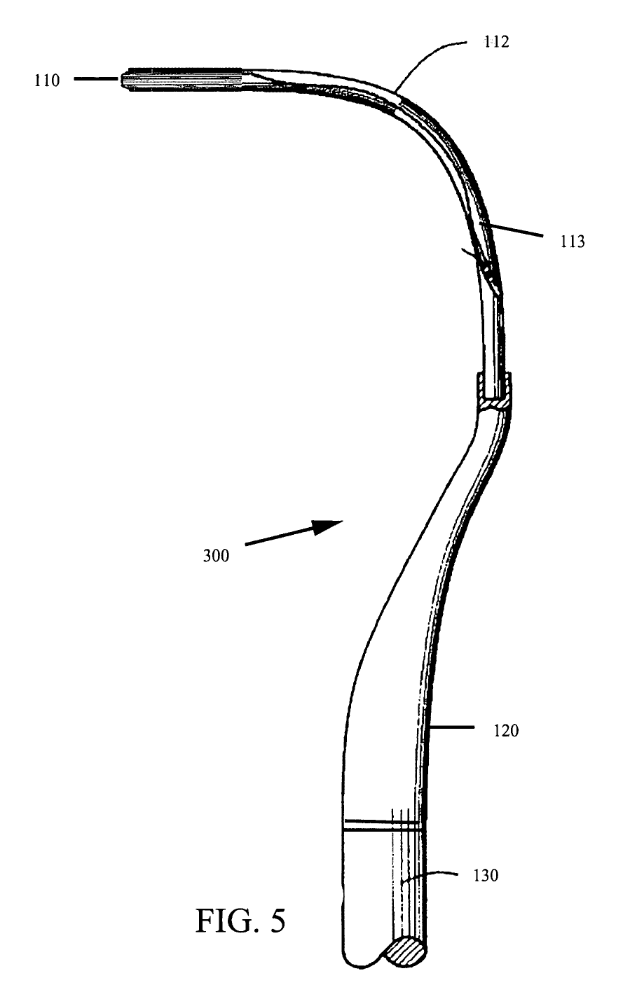

FIG. 5 illustrates yet another alternative embodiment of an insertion tool 300 which is similar to that of FIG. 3 except to illustrate that the barrel 120 may be curved in an arcuate shape for easier insertion, and may also be flexible if desired to ease insertion. Either or both features may be accomplished simply by running a flexible transmission 113 up through the body 120 and barrel 120 and connecting it to the rod 110. Otherwise, the operation is substantially the same.

In light of the foregoing it should now be apparent that the above-described insertion tools 100, 300 facilitate convenient minimally-invasive implantation of the support coil 2, and the support coil 2 effectively augments the existing sack of fibrocartilage (rather than replacing it) to reinforce the support given thereby without sacrificing any range of motion.

Having now fully set forth the preferred embodiments and certain modifications of the concept underlying the present invention, various other embodiments as well as certain variations and modifications thereto may obviously occur to those skilled in the art upon becoming familiar with the underlying concept. It is to be understood, therefore, that the invention may be practiced otherwise than as specifically set forth herein.

I claim:

1. A spinal intervertebral support coil sized to fit inside the intervertebral sac between adjacent vertebral bones of the spine and formed as a helical spring of resilient material, and further comprising a series of winding coils leading to a sharp penetrating tip for piercing a disk membrane.

2. The spinal invertebral support coil according to claim 1, wherein said series of winding coils become progressively smaller toward said sharp penetrating tip.

3. The spinal invertebral support coil according to claim 1 adapted for corkscrew insertion within an intervertebral disk sack between opposing disk bodies for additional support thereof.

4. The spinal invertebral support coil according to claim 1, wherein said support coil interposes a predetermined bias between said opposing disk bodies and yet allows a limited degree of rotational movement, torsional movement, and axial movement in a compressive direction for displacement, rotation, subluxation, flexion, and extension of the vertebral bodies.

5. The spinal intervertebral support coil of claim 1, wherein the support coil comprises a self-expanding material for expansion after insertion.

6. The spinal intervertebral support coil of claim 5, wherein said self-expanding material is water absorptive.

7. The spinal intervertebral support coil of claim 5, wherein said self-expanding material is Nitinol™.

8. The spinal intervertebral support coil of claim 1 wherein the support coil exerts a predetermined bias between the upper and lower disk bodies substantially equal to that of a healthy invertebral sack.

9. A spinal intervertebral support coil sized to fit inside the intervertebral sac between adjacent vertebral bones of the spine and formed as a helical spring of resilient material, further comprising a keyed hub attached to one end of a series of helical coils and running to a sharp penetrating tip of another end of said series of coils.

10. The spinal intervertebral support coil of claim 9 wherein said hub is keyed for engagement by an insertion tool for corkscrew insertion into an intervertebral disk sack between opposing disk bodies.

11. The spinal invertebral support coil according to claim 9, wherein said support coil interposes a predetermined bias between said opposing disk bodies and yet allows a limited degree of rotational movement, torsional movement, and axial movement in a compressive direction for displacement, rotation, subluxation, flexion, and extension of the vertebral bodies.

12. The spinal intervertebral support coil of claim 9, wherein the support coil comprises a self-expanding material for expansion after insertion.

13. The spinal intervertebral support coil of claim 12, wherein said self-expanding material is water absorptive.

14. The spinal intervertebral support coil of claim 12, wherein said self-expanding material is Nitinol™.

15. The spinal intervertebral support coil of claim 9 wherein the support coil exerts a predetermined bias between the upper and lower disk bodies substantially equal to that of a healthy invertebral sack.

16. A spinal intervertebral support system, comprising:
a coil sized to fit inside the intervertebral sac between adjacent vertebral bones of the spine and formed as a helical spring of resilient material, and further comprising a keyed hub attached to one end of a series of helical coils and running to a sharp penetrating tip of another end of said series of coils; and
an insertion tool for corkscrew insertion of said coil by keyed engagement to said hub and rotation thereof.

17. The spinal intervertebral support system according to claim 16, wherein said insertion tool further comprises a handle, a tubular sheath, and a rotating rod within said sheath.

18. The spinal intervertebral support system according to claim 17, further comprising an actuator for rotating said rod.

19. The spinal intervertebral support system according to claim 16, wherein one end of said rod is keyed to said hub.

20. The spinal intervertebral support system according to claim 16, wherein said rod is defined by at least one internal lumen for passing any one from among the group of an endoscope fiber, irrigation flow, or a tissue resection system.

21. A method for augmenting the intervertebral sac between adjacent vertebral bones of a spine, comprising a steps of:
piercing a membrane of said sac with a sharp distal tip of a helical coil; and
inserting said helical coil into said sac by rotating said coil along its helix, in a corkscrew manner, thereby interposing a predetermined bias between said opposing disk bodies and yet allowing a limited degree of rotational movement, torsional movement, and axial movement in a compressive direction for displacement, rotation, subluxation, flexion, and extension of the vertebral bodies.
